(19) United States
(12) Patent Application Publication
(10) Pub. No.: US 2026/0221785 A1
(43) Pub. Date: Jul. 30, 2026

(54) BRACELET WITH INTEGRATED ELECTRONICS

(71) Applicant: Baracoda Labs, Neuilly-sur-Seine (FR)

(72) Inventors: Thomas Serval, Neuilly-sur-Seine (FR); Arthur Quentin Eberhardt, Neuilly-sur-Seine (FR)

(21) Appl. No.: 19/143,697

(22) PCT Filed: Dec. 29, 2023
(86) PCT No.: PCT/IB2023/063390

§ 371 Date: Jun. 26, 2025

Related U.S. Application Data
(60) Provisional application No. 63/436,061, filed on Dec. 29, 2022.
(60) Provisional application No. 63/446,219, filed on Feb. 16, 2023.

(51) Int. Cl. H02J 7/35; A44C 5/00; A61B 5/00; A61B 5/024; A61B 5/11

(52) U.S. Cl. CPC H02J 7/35; A44C 5/0015; A61B 5/02416; A61B 5/02438; A61B 5/1118; A61B 5/681; A61B 2560/0204

(57) ABSTRACT

A bracelet includes a first band portion including an electronics enclosure for at least a battery, a network interface, and one or more sensors. The bracelet further includes a second band portion fixed to the first band portion. The second band portion has a first link and a first solar panel mounted to the first link. The bracelet further includes a third band portion removably coupled to the first band portion.

### CROSS-REFERENCE TO RELATED APPLICATIONS

This application claims priority to and the benefit of U.S. Provisional Patent Application No. 63/446,219, filed on Feb. 16, 2023 and U.S. Provisional Patent Application No. 63/436,061, filed on Dec. 29, 2022, each of which is hereby incorporated herein by reference in its entirety.

### FIELD OF THE INVENTION

The present invention relates generally to bracelets and/or straps with integrated electronics that can be used with analog and/or digital watches.

### BACKGROUND OF THE INVENTION

Smart watches are ubiquitous, but the aesthetics of analog watches still holds with a large number of individuals. Analog watches typically do not include “smart” features of smart watches, hence are more limited in functionality. Accordingly, it is desirable to increase functionality of analog watches.

### SUMMARY

The term embodiment and like terms, e.g., implementation, configuration, aspect, example, and option, are intended to refer broadly to all of the subject matter of this disclosure and the claims below. Statements containing these terms should be understood not to limit the subject matter described herein or to limit the meaning or scope of the claims below. Embodiments of the present disclosure covered herein are defined by the claims below, not this summary. This summary is a high-level overview of various aspects of the disclosure and introduces some of the concepts that are further described in the Detailed Description section below. This summary is not intended to identify key or essential features of the claimed subject matter. This summary is also not intended to be used in isolation to determine the scope of the claimed subject matter. The subject matter should be understood by reference to appropriate portions of the entire specification of this disclosure, any or all drawings, and each claim.

According to certain aspects of the present disclosure, a bracelet includes a first band portion including an electronics enclosure for at least a battery, a network interface, and one or more sensors. The bracelet further includes a second band portion fixed to the first band portion and a watch and a third band portion fixed to the watch and removably coupled to the first band portion. The second band portion has a first link, a first solar panel mounted to the first link, and a first flexible printed circuit board electrically coupling the first solar panel to the network interface of the first band portion. The third band portion has a band clasp configurable in an open configuration or a closed configuration. The band clasp includes a first printed circuit board assembly configured to electrically connect to the network interface of the first band portion when the band clasp is in the closed configuration and electrically disconnect from the network interface of the first band portion when the band clasp is in the open configuration.

According to certain aspects of the present disclosure, a system includes a watch and a bracelet. The bracelet includes a first band portion including an electronics enclosure for at least a battery, a network interface, and one or more sensors; a second band portion fixed to the first band portion and the watch; and a third band portion fixed to the watch and removably coupled to the first band portion. The second band portion has a first link, a first solar panel mounted to the first link, and a first flexible printed circuit board electrically coupling the first solar panel to the network interface of the first band portion. The third band portion has a band clasp configurable in an open configuration or a closed configuration. The band clasp includes a first printed circuit board assembly configured to electrically connect to the network interface of the first band portion when the band clasp is in the closed configuration and electrically disconnect from the network interface of the first band portion when the band clasp is in the open configuration.

According to certain aspects of the present disclosure, a bracelet includes a first band portion including an electronics enclosure for at least a battery, a network interface, and one or more sensors; a second band portion fixed to the first band portion and a watch; and a third band portion fixed to the watch and removably coupled to the first band portion. The second band portion has a first solar panel and an electrical conduit electrically coupling the first solar panel to the network interface of the first band portion. The third band portion has a joining section configurable in an open configuration or a closed configuration. The joining section electrically connects the third band portion to the network interface of the first band portion when the joining section is in the closed configuration and electrically disconnecting the third band portion from the network interface of the first band portion when the joining section is in the open configuration.

According to certain aspects of the present disclosure, a bracelet includes a first band portion including an electronics enclosure for at least a battery, a network interface, and one or more sensors; a second band portion fixed to the first band portion and a watch; and a third band portion fixed to the watch and removably coupled to the first band portion. The second band portion has a first solar panel, and an electrical conduit electrically coupling the first solar panel to the network interface of the first band portion.

According to certain aspects of the present disclosure, a bracelet includes a first band portion including an electronics enclosure for at least a battery, a network interface, and one or more sensors; a second band portion fixed to the first band portion; and a third band portion removably coupled to the first band portion. The second band portion has a first link, and a first solar panel mounted to the first link.

### BRIEF DESCRIPTION OF THE DRAWINGS

FIG. 1 is a first perspective view of a bracelet, according to some implementations of the present disclosure.

FIG. 2 is a second perspective view of the bracelet of FIG. 1.

FIG. 3 is a first side view of the bracelet of FIG. 1.

FIG. 4 is a second side view of the bracelet of FIG. 1.

FIG. 5 is a bottom perspective view of a first band portion of the bracelet of FIG. 1.

FIG. 6 is a top perspective view of the first band portion of FIG. 5.

FIG. 7 illustrates a flexible printed circuit board for connecting electronics of the bracelet of FIG. 1.

FIG. 8 illustrates the electronics in the first band portion of FIG. 5.

FIG. 9 illustrates example links in the bracelet of FIG. 1, according to some implementations of the present disclosure.

FIG. 10 illustrates a top perspective view of the electronics included in the first band portion of FIG. 5.

FIG. 11 illustrate a bottom perspective view of the electronics included in the first band portion of FIG. 5.

FIG. 12 illustrates an exploded view of the electronics included in the first band portion of FIG. 5.

FIG. 13A illustrates a first method of connecting links in the bracelet of FIG. 1.

FIG. 13B illustrates a perspective view of a pin assembly for connecting links in the first method of FIG. 13A.

FIG. 13C illustrates an exploded view of the pin assembly of FIG. 13B.

FIG. 13D illustrates a cross sectional view of the pin assembly of FIG. 13B.

FIG. 14 illustrates a second method of connecting links in the bracelet of FIG. 1.

FIG. 15A illustrates a third method of connecting links in the bracelet of FIG. 1.

FIG. 15B illustrates a fourth method of connecting links in the bracelet of FIG. 1.

FIG. 15C illustrates a fifth method of connecting links in the bracelet of FIG. 1.

FIG. 16A illustrates a clasp design, according to some implementations of the present disclosure.

FIG. 16B illustrates another clasp design, according to some implementations of the present disclosure.

FIG. 16C illustrates another clasp design, according to some implementations of the present disclosure.

FIG. 16D illustrates another clasp design, according to some implementations of the present disclosure.

FIG. 16E illustrates another clasp design, according to some implementations of the present disclosure.

FIG. 17A illustrates a clasp design, according to some implementations of the present disclosure.

FIG. 17B illustrates an example cross section of a clasp design, according to some implementations of the present disclosure.

FIG. 17C illustrates another example cross section of a clasp design, according to some implementations of the present disclosure.

FIG. 17D illustrates another example cross section of a clasp design, according to some implementations of the present disclosure.

FIG. 17E illustrates another example cross section of a clasp design, according to some implementations of the present disclosure.

FIG. 18A illustrates yet another clasp design, according to some implementations of the present disclosure.

FIG. 18B illustrates yet another clasp design, according to some implementations of the present disclosure.

FIG. 18C illustrates yet another clasp design, according to some implementations of the present disclosure.

FIG. 19A illustrates a further clasp design, according to some implementations of the present disclosure.

FIG. 19B illustrates a cross section of the clasp design of FIG. 19A.

FIG. 20 illustrates different buckle designs, according to some implementations of the present disclosure.

FIG. 21 illustrates yet another buckle design, according to some implementations of the present disclosure.

FIG. 22 illustrates a watch face attached to the buckle, according to some implementations of the present disclosure.

FIG. 23A is a graph showing a sample bracelet's thermoelectric generation of power over a time period, according to some implementations of the present disclosure.

FIG. 23B is a graph showing the sample bracelet's solar generation of power over the time period of FIG. 23A, according to some implementations of the present disclosure.

FIG. 23C is a graph showing battery level of the sample bracelet over the time period of FIG. 23A, according to some implementations of the present disclosure.

FIG. 23D is a graph showing activity level recorded by the sample bracelet over the time period of FIG. 23A, according to some implementations of the present disclosure.

FIG. 23E is a graph showing probability of heart rate variability measurement over the time period of FIG. 23A, according to some implementations of the present disclosure.

FIG. 24 is a bottom perspective view of a second bracelet, according to some implementations of the present disclosure.

FIG. 25 is a top perspective view of the bracelet of FIG. 24.

FIG. 26 is a bottom perspective view of a first band portion of the bracelet of FIG. 24.

FIG. 27 is a top perspective view of the first band portion of FIG. 26.

FIG. 28 is a top plan view of the first band portion of FIG. 26 showing current direction along the sides of the band.

FIG. 29 is an exploded view of the first band portion of FIG. 26, according to some implementations of the present disclosure.

FIG. 30 is a perspective view of a link on the bracelet of FIG. 24.

FIG. 31 is a perspective view of the link of FIG. 30, according to some implementations of the present disclosure.

FIG. 32 is an exploded view of the link of FIG. 31.

FIG. 33 is a perspective view of a link with a solar panel on the bracelet of FIG. 24.

FIG. 34 is an exploded view of the link with the solar panel of FIG. 33, according to some implementations of the present disclosure.

While the invention is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention as defined by the appended claims.

### DETAILED DESCRIPTION

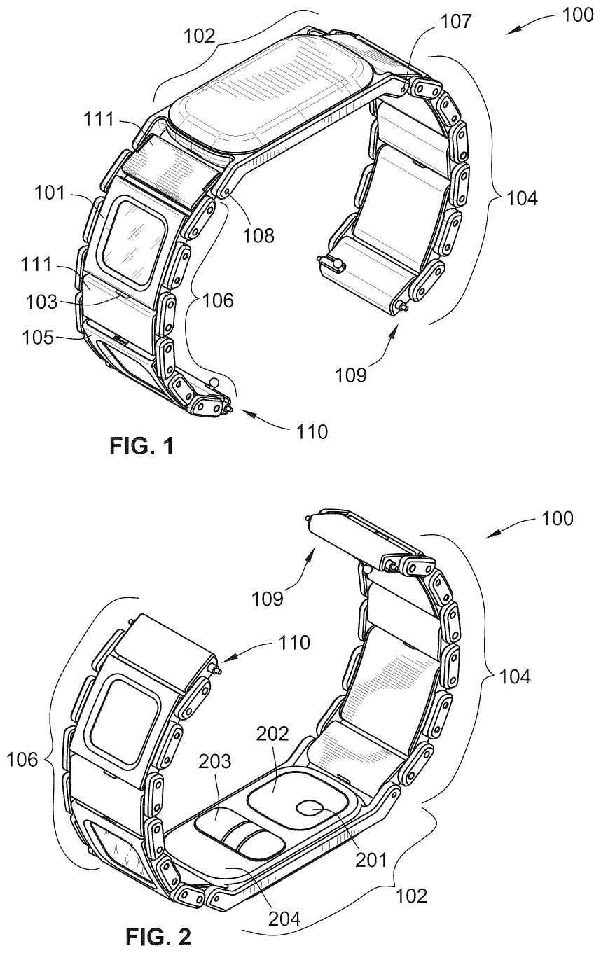

FIG. 1 is a first perspective view of a bracelet 100, according to some implementations of the present disclosure. The bracelet 100 includes a first band portion 102, a second band portion 104, and a third band portion 106. The first band portion 102 is coupled to the second band portion 104 at connection 107 and coupled to the third band portion 106 at connection 108. The first band portion 100 is separable around one of the connections 107, 108. The first band portion 100 connects to both the second band portion 104 and the third band portion 106 at the lugs of the first band portion 102. The first band portion 102 includes an electronics enclosure. The second band portion 104 and the third band portion 106 are straps with links (e.g., a second link 111, a first link 101). The first link 101 and the second link 111 can be coupled together. In some examples, the second link 111 can be replaced with a clasp that opens and closes, allowing the third band portion 106 to be disconnected from the first band portion 102. The first link 101 includes a solar panel. The end 110 of the third band portion 106 and the end 109 of the second band portion 104 can be connected to a watch. The bracelet 100 includes electronics such that the watch can be either a digital or an analog watch. Furthermore, the watch's batteries can be independent of the electronics of the electronics within the first band portion 102 and/or the solar panels in the first link 101 or a third link 105. The bracelet 100 can include one or more spaces 103 for routing a flexible printed circuit board to connect one or more solar panels and/or other electronics.

FIG. 2 is a second perspective view of the bracelet 100. The first band portion 102 includes electronics (e.g., one or more sensors). In some implementations, a transparent portion 203 of the enclosure is provided to allow a PPG sensor included in the first band portion 102 to make measurements. The transparent portion 203 includes one or more separators as illustrated for isolating emission and absorption of light. The transparent portion 203 can be plastic. In some implementations, a metal cap 201 is provided as part of the enclosure. The metal cap 201 is surrounded by non-conducting plastic 202. Since the body 204 of the first band portion 102 is metal, the non-conducting plastic 202 thermally isolates the body 204 from the metal cap 201. The metal cap 201 can be used with a temperature sensor included in the enclosure.

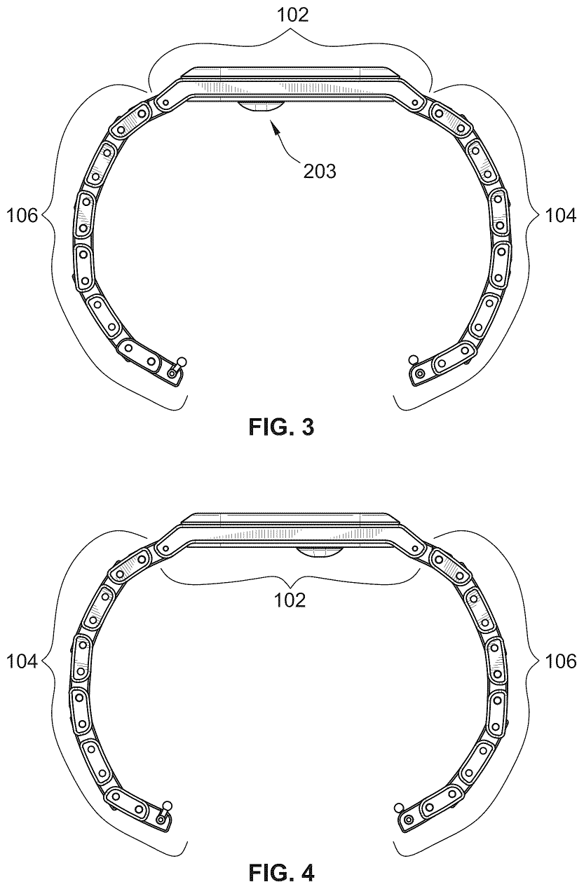

FIG. 3 is a first side view of the bracelet 100, and FIG. 4 is a second side view of the bracelet 100. The transparent portion 203 is shown to have a curvature and extrudes from the bottom of the first band portion 102. Extruding nature of the transparent portion 203 allows better contact with the skin to improve measurements.

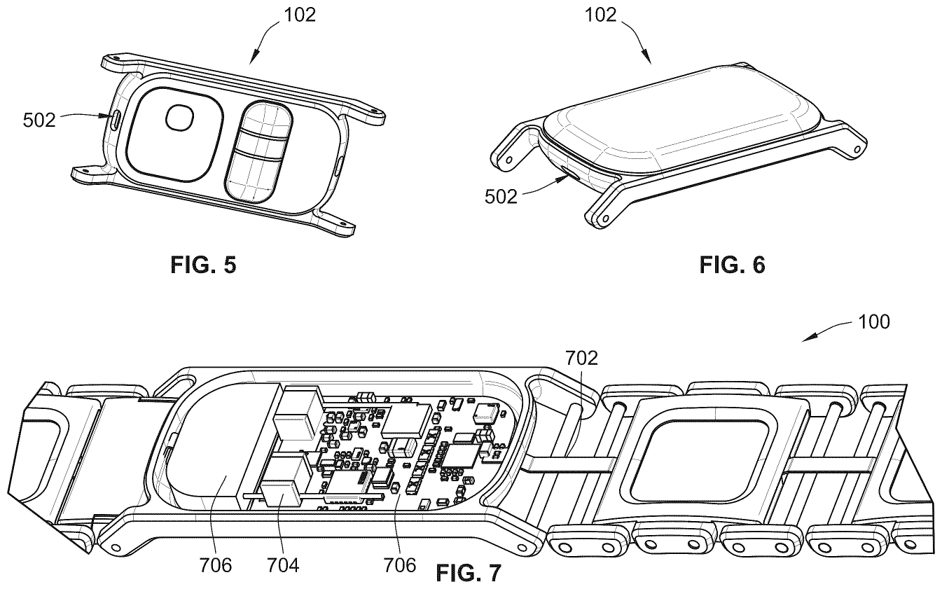

FIG. 5 is a bottom perspective view of the first band portion 102 and can include holes around the lugs for receiving a flexible printed circuit board. FIG. 6 is a top perspective view of the first band portion 102.

FIG. 7 illustrates a flexible printed circuit board 702 for connecting electronics of the bracelet 100. Links are hidden in this view to illustrate the flexible printed circuit board connection. In some implementations, the flexible printed circuit board is a single piece, and in others can be multiple flexible printed circuit boards that act as jumpers. The flexible printed circuit board functions as an electrical conduit for propagating signals and/or power throughout the bracelet 100. The first band portion 102 includes a main circuit board 706 with one or more batteries 704 and a thermoelectric generator 708. In some implementations, an energy harvester circuit is provided on the main circuit board 706. The energy harvester circuit can generate energy from motion of the enclosure, for example, if the bracelet 100 moves as a result of the wearer's limb moving.

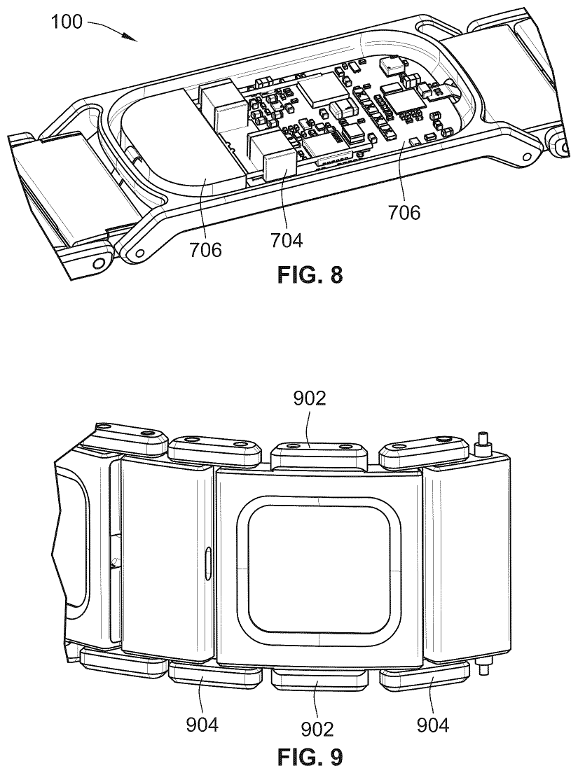

FIG. 8 illustrates the electronics in the first band portion 102. FIG. 9 illustrates example links in the bracelet 100, according to some implementations of the present disclosure. Connectors 902 can be non-removable, and connectors 904 can be removable.

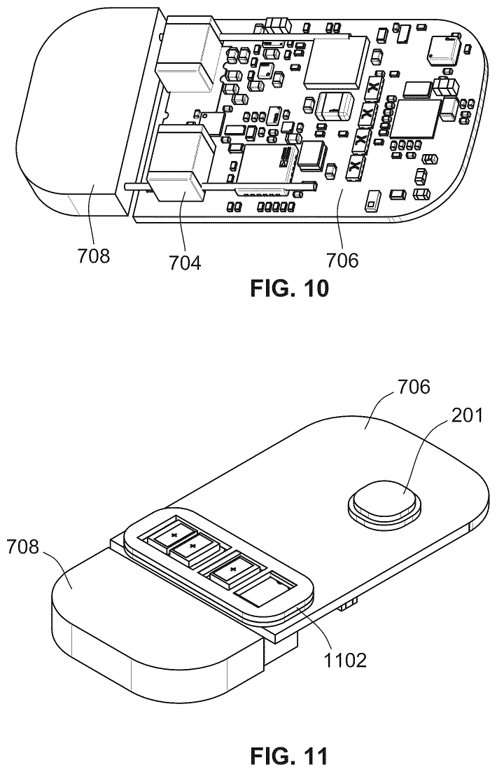

FIG. 10 illustrates a top perspective view of the electronics included in the first band portion 102 as previously described. FIG. 11 illustrate a bottom perspective view of the electronics included in the first band portion 102. A shell 1102 can be used for isolating portions of the PPG sensor. The shell 1102 can separate emitters (e.g., light emitting diodes (LEDs)) from receptors (e.g., photodiodes).

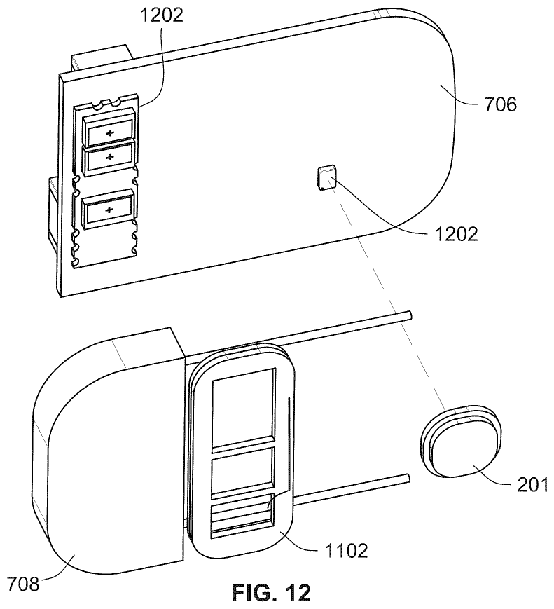

FIG. 12 illustrates an exploded view of the electronics included in the first band portion 102. The main circuit board can include a temperature sensor 1202 on the bottom that interfaces with the metal cap 201. The PPG sensor can include a daughter board 1202 with the LEDs and photosensors. In some implementations, the daughter board 1202 only contains the LEDs and photosensors (e.g., photodiodes), and analog front end is provided on the main circuit board 706.

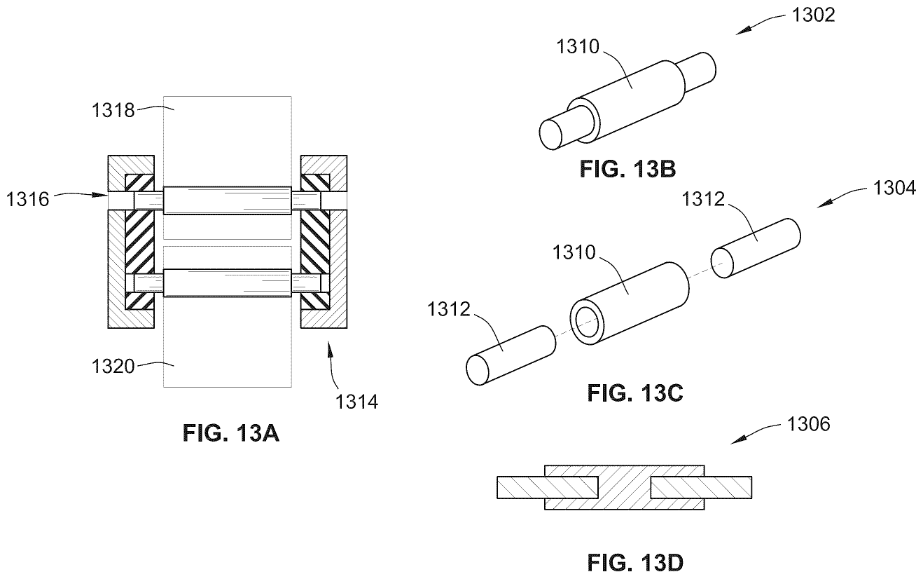

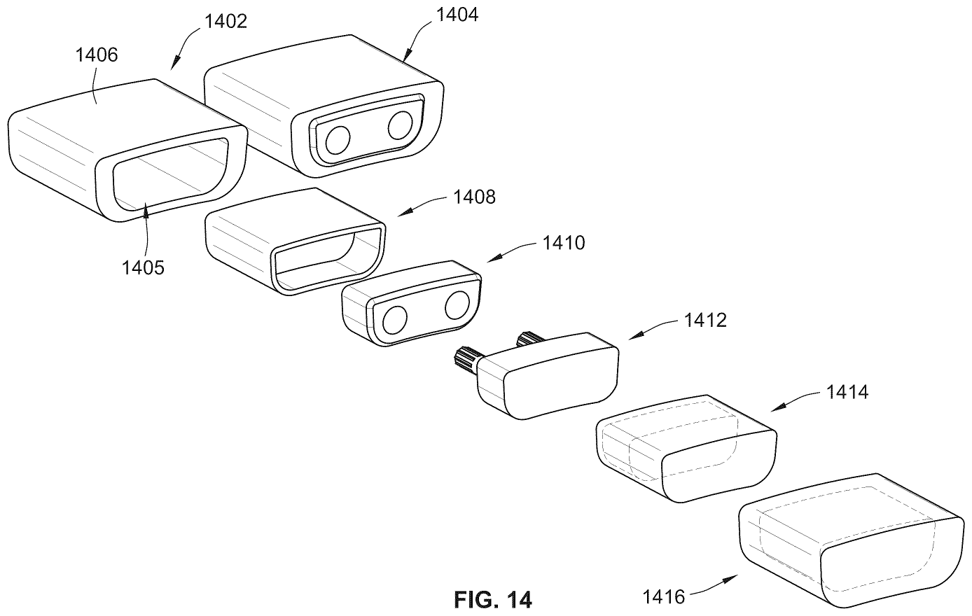

FIGS. 13A-13D illustrate a first method of connecting links in the bracelet 100 (FIG. 1). Connectors 1314 include an outer non-conducting portion (blue portion) and an inner conducting portion (brown portion). The connectors 1314 include pin holes 1316 such that when pins (e.g., pin 1312) is inserted, the pins make an electrical connection to the inner conducting portion of the conductors 1314, thereby connecting link 1318 to link 1320. The pins (e.g., 1314) are inserted on either side of a non-conducting cylinder 1310. Pumps can be provided in the non-conducting cylinder 1310, and the non-conducting cylinder 1310 can be integrated in the links 1318, 1320. The connectors 1314 essentially provide jumpers along the side of the links 1318, 1320, allowing at least two separate signals to flow along the sides of the bracelet 100. That is an electrical conduit is provided that includes two wires that are routed, via the connectors 1314 and the pins 1312 along the sides of the bracelet 100. This provides an alternative means for connecting the solar panels along the bracelet 100 when compared to the flexible printed circuit board 702 already described.

View 1302 illustrates a perspective view of two pins in a non-conducting cylinder 1310. View 1304 illustrates direction for inserting the two pins. Pushing in the two pins after insertion can activate the pump to release the pins for disconnecting the links. View 1306 illustrates a cross-section of the view 1302. In some implementations, the pin holes 1316 are provided only on one link 1318 for facilitating removal, while on the other link 1320, the pins cannot be removed.

FIG. 14 illustrates a second method of connecting links in the bracelet 100. The links 1402, 1404 are next to each other and to be connected. Each link (e.g., link 1402) can have an aesthetic cover 1406 and a molded cavity 1405 for receiving a female conductive portion 1410. In some cases, there is a non-conducting island 1408 for isolating the female conductive portion 1410 from the body of the link 1402. A male conductive portion 1412 is provided with prongs that couple the female conductive portion 1410 of the link 1402 to a female conductive portion of the link 1404. A non-conducting varnish 1414 can be provided along with an aesthetic cover 1416. The male conductive portion 1412 and the female conductive portion 1410 can be crimped together to prevent removing the links.

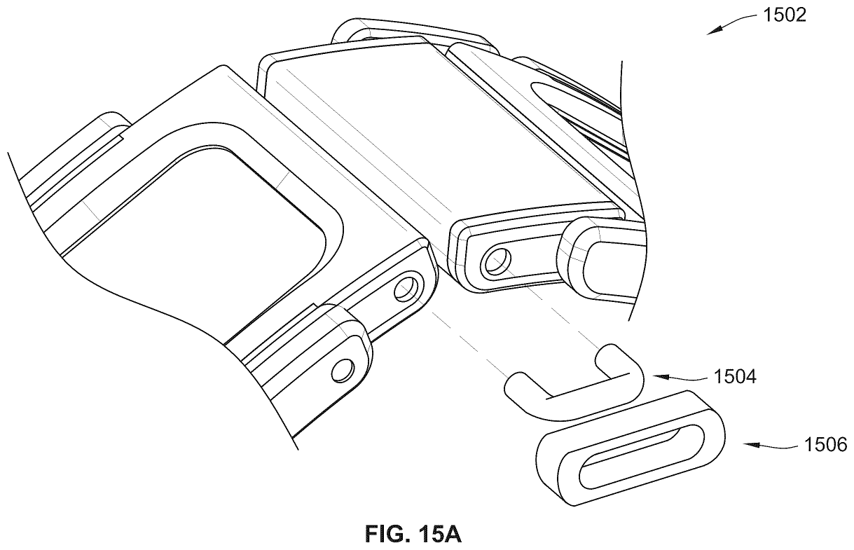

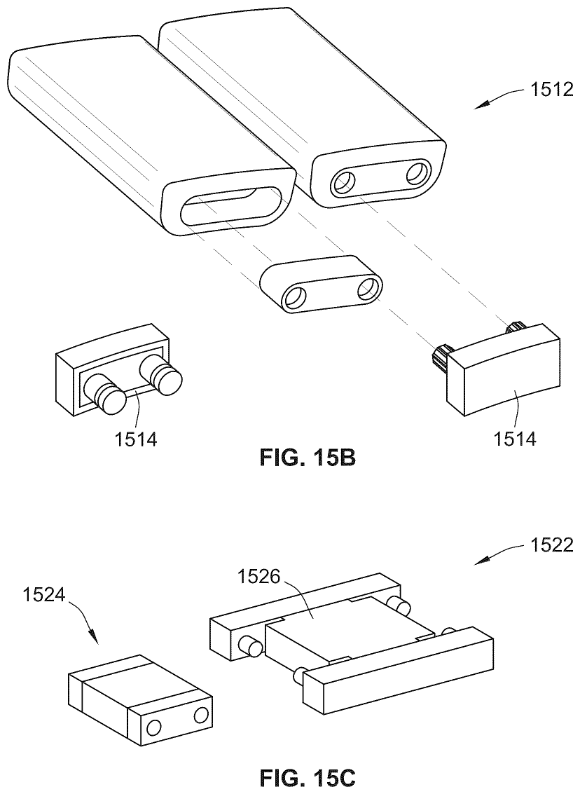

FIGS. 15A-15C illustrate third, fourth, and fifth methods of connecting links in the bracelet of FIG. 1. The third method in view 1502 is similar to FIG. 14 above except a U-shaped prong 1504 is used to couple the two links. An aesthetic cover 1506 can be used to cover the conductive prong 1504.

The fourth method in view 1512 illustrates a similar solution to FIG. 14 as well, except the blue varnishes are not provided. The male and female conductive portions can be integrated in aesthetic or non-conductive materials that form the connectors and the links.

The sixth method in view 1522 illustrates links with flexible portions 1526 that can be manipulated such that other portions 1524 can be connected and linked to a next portion.

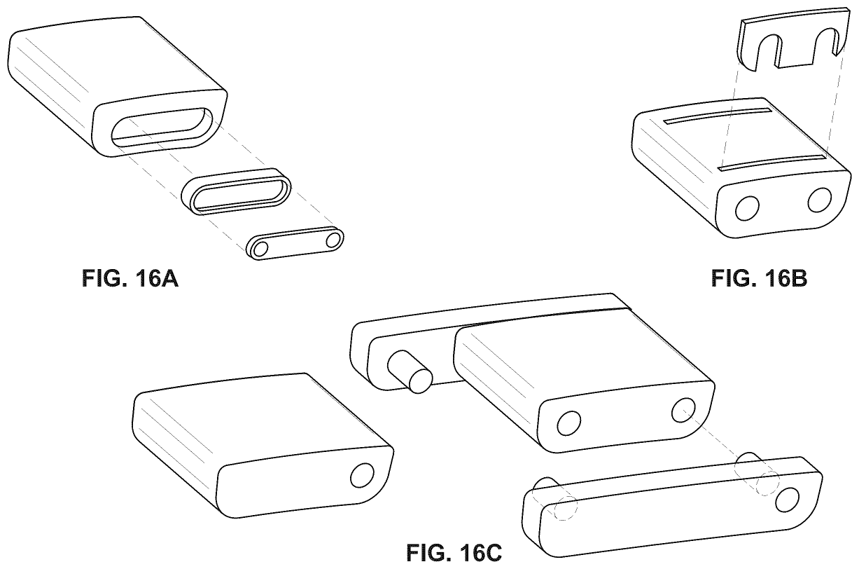

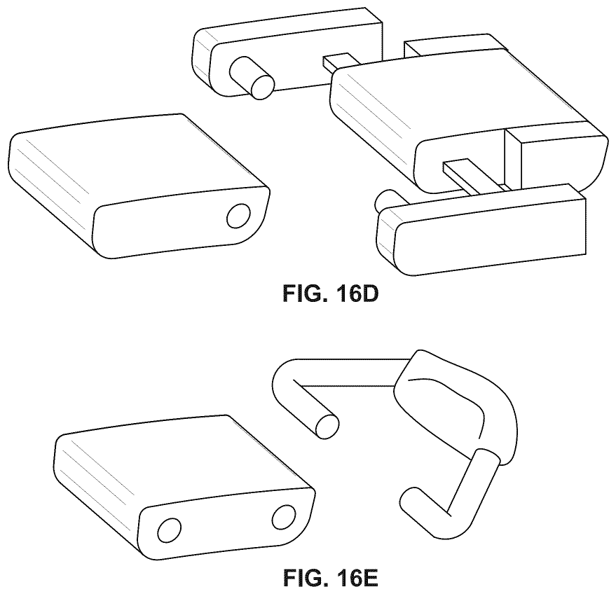

FIGS. 16A-16E illustrates different clasp designs, according to some implementations of the present disclosure. Connections can be provided along the sides of the clasps such that two wires can be routed through the straps. There can be provided a button on the clasp to open the arms of the clasp to remove the bracelet from the wrist of the user.

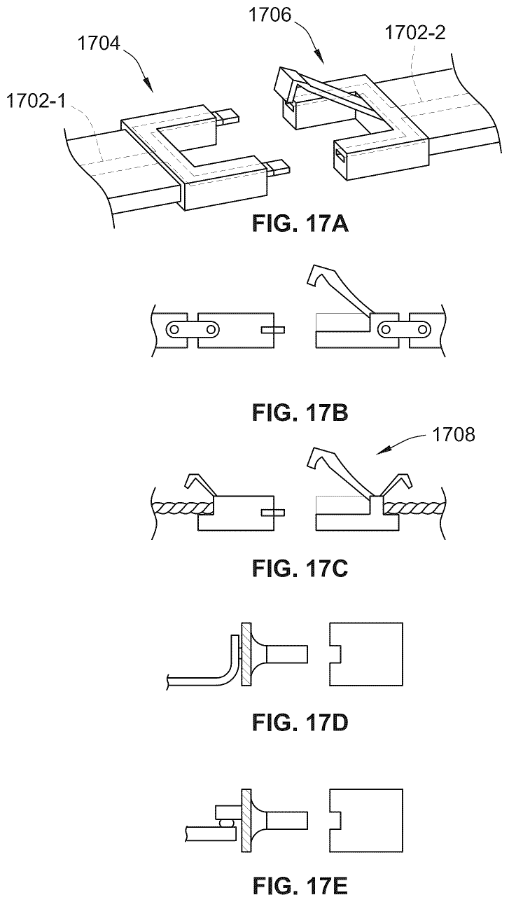

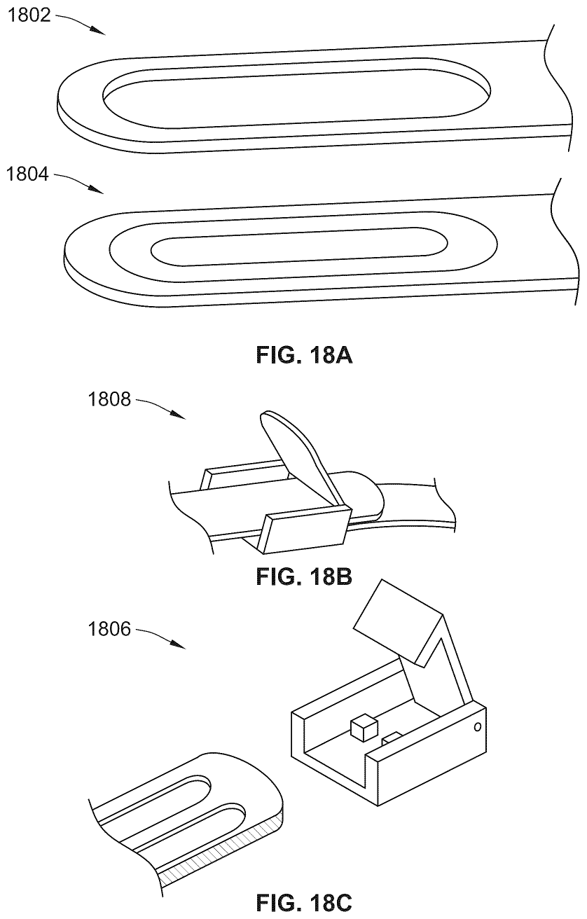

FIGS. 17A-17E illustrate another clasps design, according to some implementations of the present disclosure. The clasp can be used with a flexible strap or band. The flexible strap can include a flexible printed circuit board with two portions 1702-1 and 1702-2. The clasp has a male portion 1704 and a female portion 1706. Jaws 1708 can be provided on the female portion for securing the male portion and the female portion. Joining both portions of the clasp electrically connects the two portions 1702-1 and 1702-2 of the flexible printed circuit board.

FIGS. 18A-18C illustrate yet another clasp design, according to some implementations of the present disclosure. The solutions in FIGS. 18A-18C are similar to those of FIGS. 17A-17E. A flexible solar panel 1802 can be provided on top of the flexible bracelet design while routing can be provided on a bottom 1804 of the design. A clasp design 1806 with pins can allow connecting electrical signals of the bracelet on the bottom. Similarly, connecting on the top is possible as in view 1808.

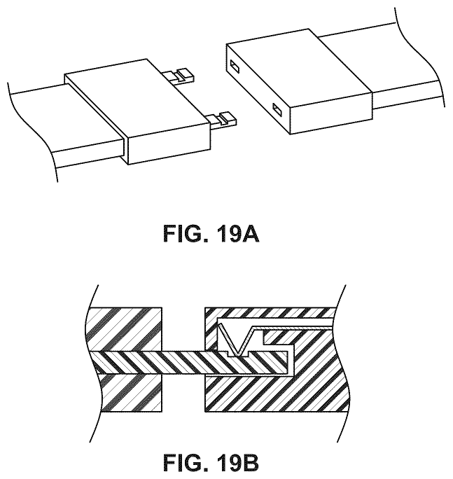

FIGS. 19A-19B illustrates a further clasp design, according to some implementations of the present disclosure. A male portion of the clasp can be received in a female portion. The female portion includes a clip for holding the male portion in place. The male portion can include a hook for interfacing with the clip.

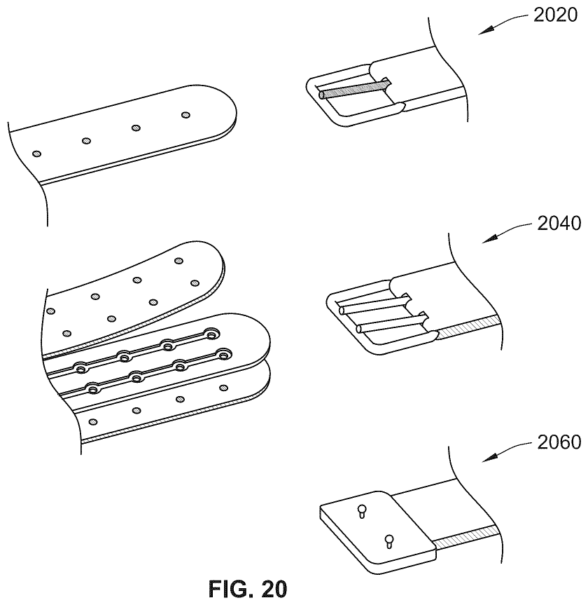

FIG. 20 illustrates different buckle designs (2020, 2040, 2060), according to some implementations of the present disclosure. In 2020, The buckle can include a frame for connecting a first electrical signal and a prong for connecting a second electrical signal. The flexible band can include holes such that when the frame is connected and the prong inserted, both sides of the flexible bands are electrically connected.

The buckle can have more than one prong as indicated in 2040. In such a case, the solar panel can include holes for receiving the prongs. The prongs can electrically connect both band portions of the bracelet.

In some implementations, the buckle includes a plurality of teeth as shown in view 2060. Each teeth can be used to route a different signal. View 2060 is similar to 2040 but in a way to avoid having holes in the solar panel because they will be on the sides of the bracelet.

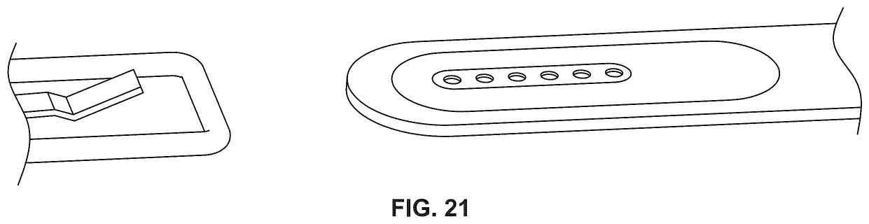

FIG. 21 illustrates yet another buckle design, according to some implementations of the present disclosure. The buckle design has all electronics on one of the band portions. That is, no need to have a connector design around the buckle for routing signals. The entirety of the band portion that includes the electronics can include a flexible solar panel that spans the entire band portion.

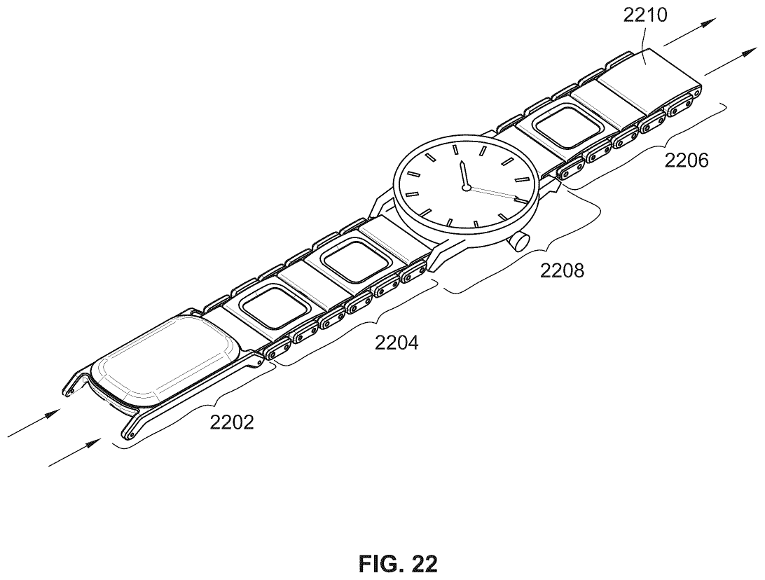

FIG. 22 illustrates a watch face 2208 attached to a bracelet, according to some implementations of the present disclosure. The bracelet includes a first band portion 2202 (similar to the first band portion 102), a second band portion 2204 (similar to the second band portion 104), and a third band portion 2206 (similar to the third band portion 106). The third band portion 2206 includes a clasp 2210 that connects to the first band portion 2202 using any of the methods discussed herein. For example, the clasp 2210 can be implemented based on FIGS. 16A to 21. The bracelet includes four solar panels.

Bracelets according to some implementations of the present disclosure can be used to trigger heart rate variability (HRV) measurements. For example a rule can be provided where HRV measurements are triggered if activity level of a user is low (e.g., 0). The activity level can be based on histogram and real-time data. The HRV measurements can be triggered if battery level is enough. HRV measurements can vary between users, so measurement energy consumption will be different for each user, thus prompting that the battery level be sufficient for the measurement performed on the user. HRV measurements are triggered when the user is wearing the bracelet.

Bracelets according to some implementations of the present disclosure can be used to trigger activity hint or reminder for the user. The activity hint can be provided on a mobile device of the user (e.g., a smartphone). The activity hint can be provided if the battery level is low, for example, so that the bracelet can capture some of the movement of the user to generate more energy. The activity hint can be provided if the activity of the current day is below a predefined threshold or a predefined level. The activity hint can be provided if data were not sent to the phone after too much time. That is, the bracelet can determine that the user is not moving enough based on the amount of data being sent to the phone over time.

Bracelets according to some implementations of the present disclosure can be used to trigger backups and/or measurement data to the phone. For example, if the batter level is high enough, then the bracelet can trigger backing up the data to the phone. If there is data to back up, then the user can trigger backing up the data on the phone. The bracelet communicates wirelessly to the phone.

Each action that consumes power (e.g., HRV measurements, data backup, activity hints) will be measured and averaged to improve the algorithm decisions over time. Each decision can be a good or bad decision. The bracelet learns from each type of decision. A good decision can be, for example, no movement during two minutes of HRV measurement, phone was present at time of backup, etc. That is, when the bracelet guesses correctly when to perform an action and the action completes successfully, then a good decision was made. In contrast, a bad decision is that the user moved during the HRV measurement, thereby nullifying the measurement, that the phone was not there for backup, etc. Histograms can be stored and updated over time to improve decisions accuracy.

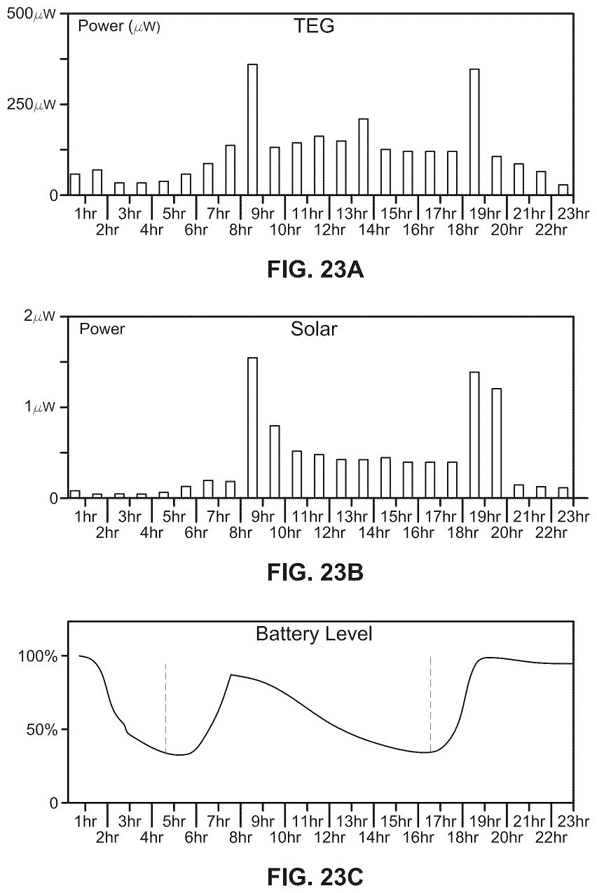

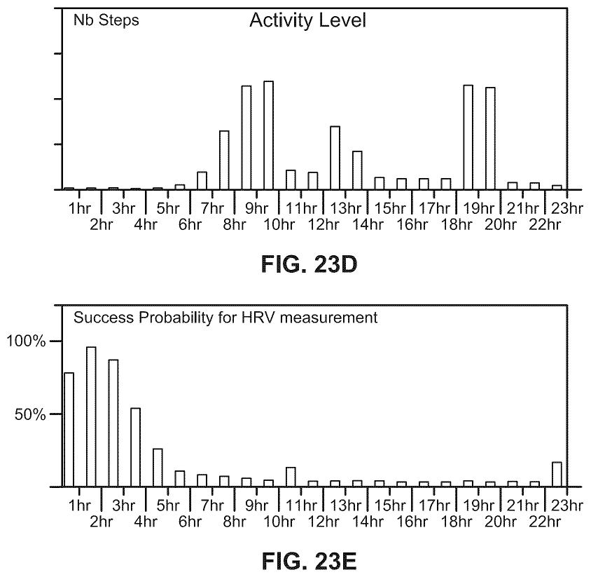

Example histograms are provided in FIGS. 23A-23D. During a 24 hour period, the histograms are provided for a 24 hour timespan.

The TEG histogram (FIG. 23A) can provide data for heat-derived power generated at each hour by the thermoelectric generator. The largest power generation times can be trip to work, trip to canteen, and trip back home in hours 8-9, 13-14, and 18-19, respectively.

The Solar histogram (FIG. 23B) can provide data for solar power generated at each hour by the solar panels. The largest power outputs can be when the user is outside, and some power output can be observed when the user is using indoor lighting. Indoor lighting levels can differ between the office and the home. For example, outside during hours 8 and 10, office lighting during hours 10 and 18, outside during hours 18 and 20, home light during hours 5 to 8 and 20 to 23, and in bed with minimal lighting between hours 0 and 5.

The battery level graph (FIG. 23C) can be used to determine when to perform measurements. If battery level is below a threshold, then no measurements are performed. Increasing battery level indicates charging. Going from hour 1 to 23, HRV measurements can be performed at 100% until the minimum battery level between hours 4 and 5. The battery can be charged between hours 5 and 8. Between hours 8 and 17, information can be sent to phone and activity can be tracked. After hour 17, the battery can be charged.

The activity level histogram (FIG. 23D) indicates number of steps per hour. In some implementations, this can include data obtained from an accelerometer. In some implementations, values in FIG. 23D indicates average number of steps expected.

Success probability of HR V measurement histogram (FIG. 23E) can be used to provide a probability distribution over the day for the best time to perform HRV measurements. That means that the bracelet will likely learn and update the histogram in order to make better decisions.

Bracelets provided according to some implementations of the present disclose can be integrated in dog collars. That is, instead of providing a bracelet for donning on the wrist of a human, the bracelet can be a dog collar, or alternatively, can couple to a decorative pendant or medal to implement a dog collar. Bracelets provided for dog collars can allow tracking activity level of dogs.

Although described as dog collars targeting dogs, any pet can benefit from such bracelet devices. For example, the bracelet may be used as a cat collar or an identifying item placed on a pet of a human.

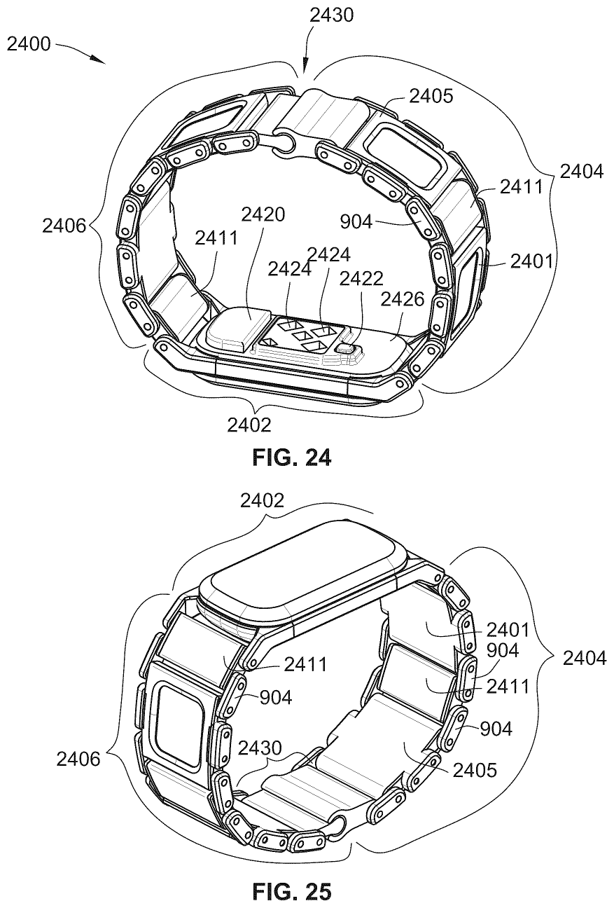

FIG. 24 is a bottom perspective view of a second bracelet 2400, according to some implementations of the present disclosure. The bracelet 2400 includes a first band portion 2402 including an electronics enclosure for at least a battery, a network interface, and one or more sensors. The bracelet 2400 further includes a second band portion 2404 fixed to the first band portion. The second band portion 2404 can couple to the first band portion 2402 in a similar manner as already described above, for example, in connection with FIGS. 13-15. The second band portion 2404 includes a first link 2405 with a first solar panel mounted to the first link 2405. The bracelet 2400 further includes a third band portion 3406. In some implementations, the third band portion 2406 is removably coupled to the first band portion as described above, for example, using band clamps. In some implementations, the third band portion 3406 includes a non-conducting coupler 2430 for removably coupling the second band portion 2404 and the third band portion 2406 such that the bracelet 2400 can be worn around a wrist of a person or a collar of an animal. In some implementations, the non-conducing coupler 2430 is not provided and the second band portion 2404 and the third band portion 2406 are fixed to a watch, in a similar manner to FIG. 22 above.

FIG. 25 is a top perspective view of the bracelet 2400. The third band portion 2406 or the second band portion 2406 can include a band clasp as described above in connection with, for example, FIG. 22. The band clasp is configurable in an open configuration or a closed configuration. The band clasp can include a first printed circuit board assembly configured to electrically connect to the network interface of the first band portion 2402 when the band clasp is in the closed configuration and electrically disconnect from the network interface of the first band portion 2402 when the band clasp is in the open configuration.

In some implementations, the second band portion 2404 and/or the third band portion 2406 further includes a second link 2401. The second link 2401 can be physically coupled via a one or more connectors (e.g., the connectors 904 of FIG. 9) to the first link 2405. A second solar panel can be mounted to the second link 2401, The one or more connectors electrically couple the second solar panel to the network interface of the first band portion 2402.

In some implementations, the second band portion 2404 and/or the third band portion 2406 further include one or more third links 2411 (e.g., the links provided in FIGS. 31-32). The third link 2411 is physically coupled via one or more connectors (e.g., the connectors 904 of FIG. 9) to the first link 2405 and/or the second link 2401. The one or more connectors electrically couple the first solar panel to the network interface.

The electronics enclosure of the first band portion 2402 includes metal or conducting portions (e.g., conducting portion 2420, metal cap 2422). The metal or conducting portions can be used with a thermoelectric generator included in the electronic enclosure and/or a thermometer. For example, the conducting portion 2420 can be used with the thermoelectric generator and the metal cap 2422 can be used for sensing a user's body temperature. The electronics enclosure can further include PPG sensors with openings 2424 in the enclosure for the PPG sensors. Although shown as openings 2424, there is a transparent window protecting the PPG sensors from environmental elements, similar to the transparent portion 203 of FIG. 2. The bracelet 2400 merely provides a different arrangement of PPG sensors compared to FIG. 2.

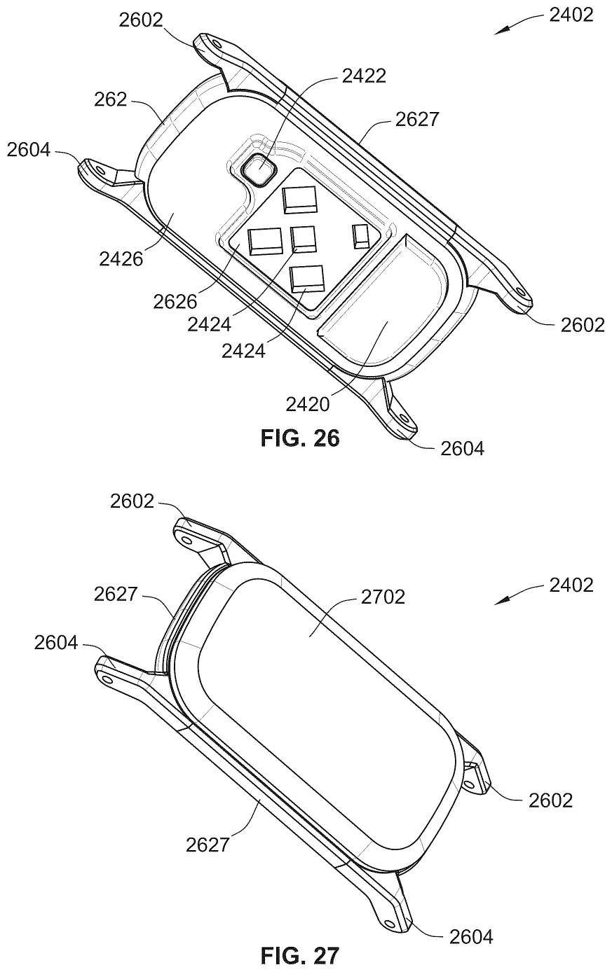

FIG. 26 is a bottom perspective view of the first band portion 2402, according to some implementations of the present disclosure. The first band portion 2402 can include a plurality of metal arms 2602, 2604. The electronic enclosure of the first band portion 2402 can include electrically and thermally insulating portions (e.g., non-conducting housing portions 2426 and sidewalls 2627 of the non-conducting housing portions 2426). Additionally, the non-conducting housing portions 2426 can include an island 2626 that protrudes into the user's skin. The protrusion of the island 2626 is provided for better skin contact and photo-isolation, and similarly the conducting portion 2420 and/or the metal cap 2422 can protrude for better skin contact during thermal measurements.

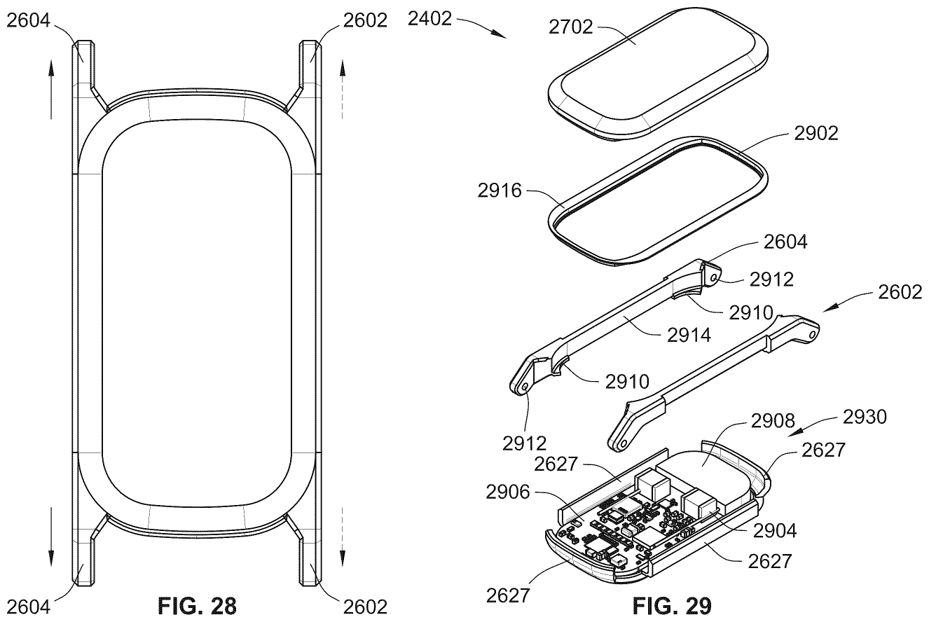

FIG. 27 is a top perspective view of the first band portion 2402. The electronic enclosure of the first band portion 2402 can have a metal cover 2702 that snaps on and off such that the electronic enclosure can be opened and closed. In some implementations, the metal cover 2702 can include only a small metal center with plastic portions surrounding the metal center. As long as part of the metal cover 2702 is thermally conductive for use by a thermoelectric generator for determining a temperature gradient for generating energy for charging the battery. FIG. 28 is a top plan view of the first band portion 2402 showing current direction along the sides of the band, according to some implementations of the present disclosure. That is, electrical current and/or voltage can be isolated along the sides of the first band portion 2402. In FIG. 28, the metal arm 2602 represents a first electrical pathway and the metal arm 2604 represents a second electrical pathway. The electrical pathways are depicted as being parallel to each other and not crossing because one metal arm is used along the side of the first band portion 2402.

In some implementations, four metal arms can be arranged so that the current crisscrosses. For example, electronics within the electronics enclosure of the first band portion 2402 can serve as a platform for connecting the four metal arms such that metal arms along the diagonal of the first band portion 2402 are electrically connected to each other.

In some implementations, the first band portion 2402 is substantially rectangular in shape and having a major axis and a minor axis. The metal arms (e.g., the metal arms 2602 and 2604) can be arranged parallel to the major axis and perpendicular to the minor axis. The metal arms can be arranged along the long side of the rectangular shape.

FIG. 29 is an exploded view of the first band portion 2402, according to some implementations of the present disclosure. The electronics enclosure of the first band portion 2402 can include a top housing portion and a bottom housing portion. The top housing portion and the bottom housing portion can be separated by a gasket 2902. The gasket 2902 is isolating and can include extended flats 2916 that further aid in isolating electrically and thermally conducting portions of the top housing portion from other parts of the first band portion 2402. The top housing portion includes the metal cover 2702, and the bottom housing portion includes non-conducting housing portions (e.g., non-conducting housing portions 2426, sidewalls 2627, island 2626) and already described conducting portions (e.g., metal cap 2422, conducting portion 2420). In some implementations, the conducting portions 2422, 2420 are not part of the bottom housing portion and are metal parts that project through an opening in the bottom housing portion.

In some implementations, the bottom housing portion receives a plurality of metal arms 2602, 2604. The metal arms 2602, 2604 can include holes 2912 for coupling the first band portion 2402 to the second band portion 2404 (FIGS. 24 and 25) and the third band portion 2406 (FIGS. 24 and 25). The metal arms 2602, 2604 electrically couple the first band portion 2402 to the second band portion 2404 and the third band portion 2406. In some implementations, the metal arms 2602, 2604 include protrusions 2910 for connecting the metal arms 2602, 2604 to a printed circuit board 2906 within the electronics enclosure of the first band portion 2402. The protrusions 2910 can reside underneath the printed circuit board 2906 making contact to a metal pad on the printed circuit board 2906. The metal arms 2602, 2604 can include extended portions 2914 for interfacing with interior of the sidewalls 2627. The extended portions 2914 can reside in channels along the sidewalls 2627 when the first band portion 2402 is assembled. Each of the metal arms 2602, 2604 represents a separate electrical potential, thus requiring electrically separating the metal arms 2602, 2604 to prevent shorting these two electrical potentials.

In some implementations, the first band portion 2402 further includes a thermoelectric generator 2908 disposed within the electronics enclosure. The thermoelectric generator 2908 is configured to absorb heat from the electronics enclosure to produce electricity from the temperature difference between a skin of the user (obtained via the conducting portion 2420) and an ambient temperature (obtained via the metal cover 2702). In some implementations, the first band portion 2402 further includes an energy harvester disposed within the electronics enclosure on the printed circuit board 2906. The energy harvester is configured to generate energy from a sensed movement of the electronics enclosure. In some implementations, the first band portion 2402 further includes one or more batteries 2904. In some implementations, the batteries 2904 can be charged via the thermoelectric generator 2908, one or more solar panels, and/or the energy harvester.

In some implementations, the first band portion 2402 includes one or more sensors. The one or more sensors include (i) a light generator configured to produce light waves that escape the electronics enclosure, (ii) a photodetector configured to absorb light waves entering the electronics enclosure to generate light ratio data, (iii) a temperature sensor configured to generate temperature data associated with a skin of the user and/or an environment, (iv) a gyroscope configured to generate orientation data associated with a positioning of the electronics enclosure, (v) an accelerometer configured to generate acceleration data, (vi) an inertial measurement unit (IMU) configured to generate position and/or velocity data, or (vii) any combination of (i)-(vi).

In some implementations, the gasket 2902 can be skipped if the metal cover 2702 includes plastic exterior (i.e., outer rims of the metal cover 2702) and has a metal center as discussed earlier in connection with FIG. 27. The plastic exterior can serve as an insulator to prevent shorting of the two metal arms 2602, 2604.

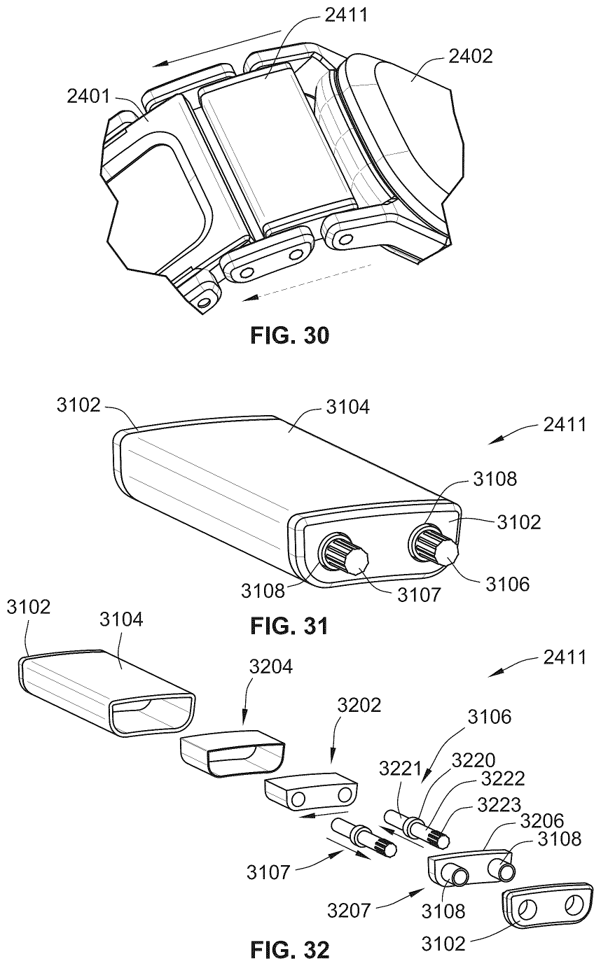

FIG. 30 is a perspective view of the third link 2411 on the bracelet 2400. The third link 2411 is provided to have a rectangular cross section, facilitating electrical current flow along the short side of the rectangular third link 2411. FIG. 31 is a perspective view of the third link 2411 when disconnected from the bracelet 2400, according to some implementations of the present disclosure. The third link 2411 includes endcaps 3102 that engage with an outer envelope 3104 of the third link 2411.

FIG. 32 is an exploded view of the third link 2411, according to some implementations of the present disclosure. The third link 2411 can further include a non-conducting island 3204 separating a female conducting portion 3202 and male conducting portions 3106, 3107 from the outer envelope 3104. The male conducting portions 3106, 3107 can include widened center regions 3220 that act as stoppers for the female conducting portion 3202 and electrical isolator 3207. The electrical isolator 3207 includes a flat portion 3206 and cylindrical portions 3108. The cylindrical portions 3108 interface with the endcaps 3102 and electrically isolate the endcaps 3102 from the male conducting portions 3106, 3107. The electrical isolator 3207 can be made from plastic. One end 3221 of the male conducting portions 3106, 3107 interfaces with the female conducting portion 3202, and a second end 3222 of the male conducting portions 3106, 3107 interfaces with the electrical isolator 3207. The second end 3222 of the male conducting portions 3106, 3107 can include patterns 3223 for promoting a frictioned grip between the male conducting portions 3106, 3107 with one or more connectors (e.g., the connectors 904 of FIG. 9).

FIGS. 31 and 32 provide the third link 2411 having conductive outer envelopes 3104 and endcaps 3102. In some implementations, the design of FIGS. 31 and 32 can be simplified if the outer envelope 3104 is not conductive. For example, there would be no need for using the island 3204 for electrical isolation.

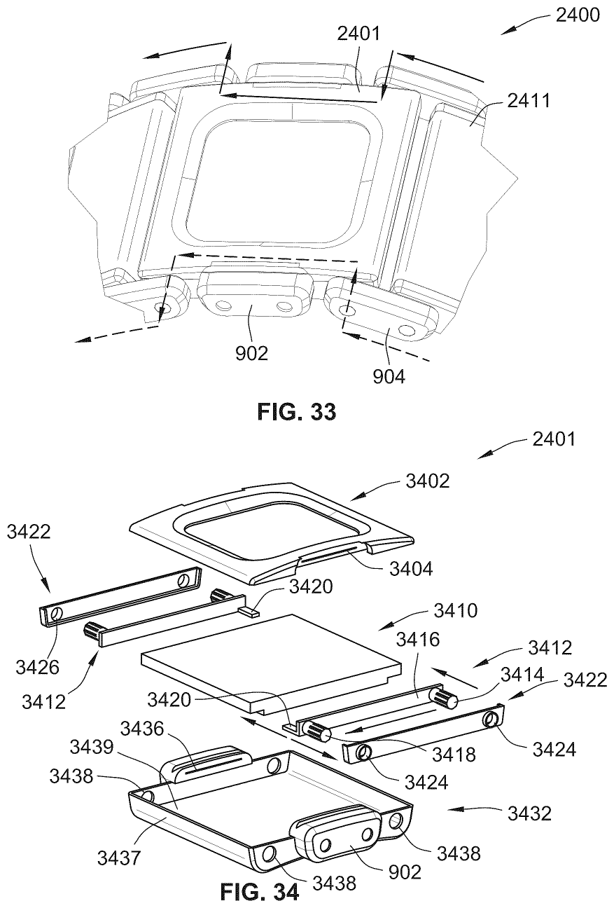

FIG. 33 is a perspective view of the first link 2401 with the solar panel on the bracelet 2400. FIG. 33 shows current flow along the band portion. The first link 2401 can allow electrical conduction along the first link as indicated by the arrows.

FIG. 34 is an exploded view of the first link 2401 with the solar panel (e.g., a first solar panel 3410), according to some implementations of the present disclosure. The first link 2401 can include a first link cover 3402 with an opening for allowing light to reach the solar panel 3410. The first link 2401 further includes a first link tray 3432 configured to receive the first link cover 3402. The first link tray 3432 has an interior for receiving the first solar panel 3410. The first link cover 3402 includes locking protrusions 3404 that can engage guides 3436 in the first link tray 3432.

The fist link 2401 further includes a pair of side conduits 3412 coupled to the first link tray 3432. The pair of side conduits 3412 is connected to the first solar panel 3410 and configured to electrically couple the first solar panel 3410 to the network interface in the first band portion 2402 (FIG. 24). Cylindrical handle portions 3414, 3418 of the side conduits 3412 can project through holes 3438 in the first link tray 3432. Flat portions 3416 of the side conduits 3412 can ride along the side of the first solar panel 3410. The side conduits 3412 can include a first side conduit that is electrically connected to a positive terminal of the first solar panel 3410 and a second side conduit that is electrically connected to a negative terminal of the first solar panel 3410.

Similar to FIG. 32, the first link 2401 can further include insulating separators 3422 configured to electrically isolate the pair of side conduits 3412 from the first link tray 3432. The insulating separators 3422 can include cylindrical portions 3424 for isolating the cylindrical nandle portions 3414 from the first link tray 3432. The side conduits 3412 can include one or more protrusions 3420 configured to connect to the first solar panel 3410. The one or more protrusions 3420 can hover above a floor 3439 of the first link tray 3432 and are electrically isolated from the floor 3439 of the first link tray 3432. An air gap for the isolation can be defined by a thickness 3436 of the insulating separators 3422. In some implementations, instead of an air isolation between the one or more protrusions 3420 and the floor 3439, one or more isolating pads can be placed in that air gap to electrically separate the floor 3439 and the one or more protrusions 3420. In some implementations, an isolating tape can be placed on the floor 3439 when using the air gap for further protection from shorts.

Many of the embodiments provided can be combined with any other embodiment. For example, one or more elements or aspects or steps, or any portion(s) thereof, from one or more of any of claims 1 to 102 below can be combined with one or more elements or aspects or steps, or any portion(s) thereof, from one or more of any of the other claims 1 to 102 or combinations thereof, to form one or more additional implementations and/or claims of the present disclosure.

1. A bracelet comprising:
a first band portion including an electronics enclosure for at least a battery, a network interface, and one or more sensors;
a second band portion fixed to the first band portion and a watch, the second band portion having:
a first link;
a first solar panel mounted to the first link; and
a first flexible printed circuit board electrically coupling the first solar panel to the network interface of the first band portion; and
a third band portion fixed to the watch and removably coupled to the first band portion, the third band portion having:
a band clasp configurable in an open configuration or a closed configuration, the band clasp including a first printed circuit board assembly configured to electrically connect to the network interface of the first band portion when the band clasp is in the closed configuration and electrically disconnect from the network interface of the first band portion when the band clasp is in the open configuration.

2. The bracelet of claim 1, wherein the second band portion further includes:
a second link, the second link physically coupled via a first connector to the first link; and
a second solar panel mounted to the second link,
wherein the first flexible printed circuit board further electrically couples the second solar panel to the network interface of the first band portion.

3. The bracelet of claim 1, wherein the first flexible printed circuit board is (i) a single printed circuit board, (ii) multiple electrically coupled printed circuit boards, or (iii) multiple electrically coupled wires.

4. The bracelet of claim 1, wherein the third band portion further includes a third link physically coupled via a second connector to a fourth link, the third band portion having a third solar panel mounted to the third link, and a second flexible printed circuit board electrically coupling the third solar panel to the network interface of the first band portion via the band clasp.

5. The bracelet of claim 1, wherein the first band portion further includes a thermoelectric generator disposed within the electronics enclosure, the thermoelectric generator configured to absorb heat from the electronics enclosure to produce electricity from the temperature difference between a skin of the user and an ambient temperature.

6. The bracelet of claim 1, wherein the first band portion further includes an energy harvester disposed within the electronics enclosure, the energy harvester configured to generate energy from a sensed movement of the electronics enclosure.

7. The bracelet of claim 1, wherein the one or more sensors include (i) a light generator configured to produce light waves that escape the electronics enclosure, (ii) a photodetector configured to absorb light waves entering the electronics enclosure to generate light ratio data, (iii) a temperature sensor configured to generate temperature data associated with a skin of the user and/or an environment, (iv) a gyroscope configured to generate orientation data associated with a positioning of the electronics enclosure, (v) an accelerometer configured to generate acceleration data, (vi) an inertial measurement unit (IMU) configured to generate position and/or velocity data, or (vii) any combination of (i)-(vi).

8. The bracelet of claim 1, wherein the electronics enclosure further includes at least one transparent window.

9. The bracelet of claim 8, wherein two transparent windows are provided, the two transparent windows separated by a distance d; and optionally, wherein a first one of the transparent windows is proximate to a light generator and a second one of the transparent windows is proximate to a photodetector.

10. The bracelet of claim 1, wherein the electronics enclosure further includes a processor and a non-transitory computer-readable medium, the non-transitory computer-readable medium storing instructions such that when executed configures the processor to:
determine an available energy level.

11. The bracelet of claim 10, wherein the processor is further configured to:
set, based on the available energy level, (i) a measurement time for at least one of the one or more sensors, (ii) a sleep time for at least one of the one or more sensors, (iii) a communication time for the network interface, (iv) a priority of making measurements by the one or more sensors, (v) a movement alert for a user of the bracelet to increase activity, or (vi) any one of (i)-(v).

12. The bracelet of claim 10, wherein the processor is further configured to:
trigger a backup of measurement data based at least in part on the determined available energy level and amount of measurement data accumulated.

13. The bracelet of claim 10, wherein the processor is further configured to:
determine a communication habit of the network interface with a mobile device based on historical data associated with previous communications between the network interface and the mobile device; and
schedule a time to communicate with the mobile device based on the communication habit.

14. (canceled)

15. The bracelet of claim 10, wherein the processor is further configured to:
trigger heart rate variability measurement based at least in part on an activity level of the user, the available energy level of the battery, and the user wearing the bracelet.

16. (canceled)

17. The bracelet of claim 10, wherein the processor is further configured to:
trigger a movement alert for a user of the bracelet to increase activity based at least in part on (i) an energy level of the battery, (ii) an activity level of the user is below a predefined activity level, (iii) a time split between a last communication with a mobile device of the user, or (iv) any one of (i)-(iii).

18-20. (canceled)

21. The bracelet of claim 1, wherein the first band portion further includes a main circuit board integrating at least the network interface and a temperature sensor, the temperature sensor located on a bottom side of the main circuit board opposite a top side of the main circuit board including the network interface.

22-26. (canceled)

27. A system comprising:
a watch; and
a bracelet including
a first band portion including an electronics enclosure for at least a battery, a network interface, and one or more sensors;
a second band portion fixed to the first band portion and the watch, the second band portion having:
a first link;
a first solar panel mounted to the first link; and
a first flexible printed circuit board electrically coupling the first solar panel to the network interface of the first band portion; and
a third band portion fixed to the watch and removably coupled to the first band portion, the third band portion having:
a band clasp configurable in an open configuration or a closed configuration, the band clasp including a first printed circuit board assembly configured to electrically connect to the network interface of the first band portion when the band clasp is in the closed configuration and electrically disconnect from the network interface of the first band portion when the band clasp is in the open configuration.

28. The system of claim 27, wherein the second band portion further includes:
a second link, the second link physically coupled via a first connector to the first link; and
a second solar panel mounted to the second link,
wherein the first flexible printed circuit board further electrically couples the second solar panel to the network interface of the first band portion.

29-84. (canceled)

85. The bracelet of claim 1, wherein the electronics enclosure of the first band portion has a top housing portion and a bottom housing portion, the top housing portion and the bottom housing portion being separated by a gasket, and the top housing portion including a metal cover and the bottom housing portion including non-conducting housing portion.

86-96. (canceled)

97. The bracelet of claim 1, wherein the first link includes:
a first link cover with an opening;
a first link tray configured to receive the first link cover, the first link tray having an interior for receiving the first solar panel; and
a pair of side conduits coupled to the first link tray, the pair of side conduits connected to the first solar panel and configured to electrically couple the first solar panel to the network interface.

98-102. (canceled)
